(19) United States
(12) Patent Application Publication
(10) Pub. No.: US 2021/0260104 A1
(43) Pub. Date: Aug. 26, 2021

(54) SOLUTION FOR PROLONGING OPEN TIME OF FERTILIZATION HOLE OF FISH EGGS AND METHOD FOR INTRODUCING EXOGENOUS GENE THROUGH FERTILIZATION HOLE

(71) Applicant: Freshwater Fisheries Research Center of China Academy of Fisheries Sciences, Wuxi (CN)

(72) Inventors: Zheming Cao, Wuxi (CN); Jun Qiang, Wuxi (CN); Pao Xu, Wuxi (CN)

(21) Appl. No.: 17/030,023

(22) Filed: Sep. 23, 2020

(30) Foreign Application Priority Data
Feb. 25, 2020 (CN) 202010116099.7

(51) Int. Cl. A61K 33/14; A61K 33/10; A61K 31/7004; A61P 15/08

(52) U.S. Cl. CPC A61K 33/14; A61P 15/08; A61K 31/7004; A61K 33/10

(57) ABSTRACT

The present application provides a solution for prolonging open time of fertilization hole of fish eggs and a method for introducing exogenous gene through fertilization hole, belonging to the technical field of research and production of transgenic fish, wherein the solution takes water as a solvent and includes 6 g of sodium chloride, 0.15 g of potassium chloride, 0.1 g of calcium chloride, 0.1 g of sodium bicarbonate, 0.1 g of sodium dihydrogen phosphate and 1 g of glucose per liter. The solution provided by the present application can prolong the open time of the fertilization hole of the fish eggs, so that the exogenous genes can enter the fish eggs directly.

### CROSS REFERENCE TO RELATED APPLICATION

This application claims the priority of Chinese Patent Application No. 202010116099.7 entitled “Solution for prolonging open time of fertilization hole of fish eggs and method for introducing exogenous gene through fertilization hole” filed with China National Intellectual Property Administration on Feb. 25, 2020, which is incorporated herein by reference in its entirety.

### INCORPORATION BY REFERENCE

A Sequence Listing appears following page six of the present application and is incorporated herein by reference in its entirety.

### TECHNICAL FIELD

The present application belongs to the technical field of research and production of transgenic fish, especially relating to a solution for prolonging open time of fertilization hole of fish eggs and a method for introducing exogenous gene through fertilization hole.

### BACKGROUND ART

At present, common introduction techniques include microinjection method, sperm carrying method, electroporation and the like. Among them, the microinjection method is currently the most commonly used and a more effective gene introduction method. Microinjection is the first adopted transgenic method, which has been widely used and achieved good results. The method comprises the steps that, a certain amount of exogenous genes are injected into the fertilized eggs by using a glass fine needle with a diameter of several microns, and the injected fertilized eggs are developed into fry in normal saline at room temperature.

The microinjection has the disadvantages that the integrity of the fish egg shells is damaged, and the egg mortality rate after microinjection is relatively high; the microinjection has strict requirements on injection time, and the injection must be performed between 2 cell stage to 16 cell stage after fertilization, otherwise chimera is easy to generate; the microinjection efficiency is not high, i.e. only a few of fish eggs can be operated at one time; it is technical difficult for the operators.

The sperm carrying method has the disadvantages that the binding efficiency of sperms and exogenous DNA is not high and the binding is unstable, so that the positive proportion of transgenic fish in different batches is unstable. The exogenous DNA is more likely to be degraded after being introduced into a fertilized egg through sperm, and is not easy to integrate into the genome.

The fertilization hole is located in the shell of fish egg, which is the channel for sperm to enter fish egg. The size of the fertilization hole is equivalent to that of the sperms, and is generally dozens to hundreds of times of the diameter of the introduced exogenous DNA, so that the exogenous DNA can easily enter unfertilized egg through the fertilization hole. However, the shell of fish egg will absorb water and expand quickly after the fish egg enters the natural environment, and the fertilization hole disappears.

### SUMMARY OF THE INVENTION

In view of the above, the purpose of the present application is to provide a solution for prolonging open time of fertilization hole of fish eggs and a method for introducing exogenous gene through fertilization hole, and the solution provided by the present application can prolong the open time of the fertilization hole of the fish eggs, which facilitates the introduction of exogenous genes into fish eggs.

In order to achieve the purpose of the application, the following technical scheme is provided:

The present application provides a solution for prolonging the open time of fertilization hole of fish eggs, the solution takes water as a solvent and includes 6 g of sodium chloride, 0.15 g of potassium chloride, 0.1 g of calcium chloride, 0.1 g of sodium bicarbonate, 0.1 g of sodium dihydrogen phosphate and 1 g of glucose per liter.

In some embodiments, the water is distilled water.

The application also provides a method for introducing an exogenous gene through fertilization hole, which includes the steps of:

1) mixing the solution described in the above technical scheme with the cationic liposome (Lipofecter) and exogenous genes to obtain a mixed solution;

2) mixing the mixed solution obtained in step 1) with fish eggs, and oscillating for 10˜30 min.

In some embodiments, the volume ratio of the solution to the cationic liposome (Lipofecter) in step 1) is 1:0.1.

In some embodiments, the ratio of the volume of the mixed solution to the number of the fish eggs in step 2) is 4 ml:1000.

In some embodiments, the oscillating time in step 2) is 20 min.

In some embodiments, the oscillating speed in step 2) is 60 rpm.

In some embodiments, the fish eggs include eggs of tilapia and carp.

The application provides a solution for prolonging the open time of fertilization hole of fish eggs, which takes water as a solvent and includes 6 g of sodium chloride, 0.15 g of potassium chloride, 0.1 g of calcium chloride, 0.1 g of sodium bicarbonate, 0.1 g of sodium dihydrogen phosphate and 1 g of glucose per liter. The solution provided by the application can prolong the open time of the fertilization hole of the fish eggs, so that the exogenous genes can enter the fish eggs directly. In the present application, the composition of the solution is close to that of fish body fluid, and the salt concentration is equivalent, so that the fish eggs can remain the original state without absorbing water in the solution, and the fertilization holes are always open.

Compared with the prior art, the application has the following advantages:

1. The damage to fish eggs is very small compared with microinjection;

2. The technical requirements for the operator are relatively lower compared with microinjection;

3. A large number of fish eggs can be treated at one time compared with microinjection;

4. The introduced exogenous DNA is not easily degraded compared with microinjection;

5. The introduction effect is stable and the positive rate of offspring is higher compared with sperm carrying method.

### BRIEF DESCRIPTION OF THE DRAWINGS

FIG. 1 is an electrophoresis spectrum of PCR product after the 1^{st} amplification;

FIG. 2 is an electrophoresis spectrum of PCR product after the 2^{nd} amplification;

FIG. 3 is a pattern of DANA retroposon amplified by PCR.

### DETAILED DESCRIPTION OF THE EMBODIMENTS

The present application provides a solution for prolonging open time of fertilization hole of fish eggs, the solution takes water as a solvent and includes 6 g of sodium chloride, 0.15 g of potassium chloride, 0.1 g of calcium chloride, 0.1 g of sodium bicarbonate, 0.1 g of sodium dihydrogen phosphate and 1 g of glucose per liter. In some embodiments, the water is preferably distilled water.

The application also provides a method for introducing exogenous gene through fertilization hole, which includes the steps of:

1) mixing the solution in the above technical scheme with the cationic liposome (Lipofecter) and exogenous genes to obtain a mixed solution;

2) mixing the mixed solution obtained in step 1) with fish eggs, and oscillating for 10˜30 min.

In some embodiments, the volume ratio of the solution to the cationic liposome (Lipofecter) is preferably 1:0.1. In some embodiments, the function of the cationic liposome is to form a polymer with an exogenous DNA sequence in a salt solution, which is easier to enter the fish eggs.

In some embodiments, there is no particular limitation on the sequence of the exogenous genes, any gene sequence will do, and the dosage of the exogenous gene is not particularly limited.

In some embodiments, the cationic liposome is preferably Lipofecter, and there is no particular limitation on the source of the Lipofecter, a commercially available product will do.

In some embodiments, the ratio of the volume of the mixed solution to the number of fish eggs is preferably 4 ml:1000. In some embodiments, the fish eggs are preferably eggs of tilapia and carp. The fish eggs from the female fish to be spawned are subjected to artificial maturation and are squeezed out manually, and the fish eggs may not expose to moisture. In some embodiments, the oscillating time is preferably 20 min, and the oscillating speed is preferably 60 rpm.

In some embodiments, the obtained mixed solution is mixed with fish eggs and oscillated for 10˜30 min, then the mixed solution is mixed with sperms evenly, the sperms are activated by adding clear water after being oscillated for 3˜5 min, after that the fertilization process is completed by oscillating for another 3˜5 min, and the fertilized eggs are obtained. In some embodiments, the fertilized eggs are hatched in a natural state. The fertilization holes are opened after the fish eggs mature in the body of the female fish, and close quickly when entering the low-salt solution, so the sperms may not enter the fish eggs. The fish eggs treated with the solution provided by the present application may still be fertilized, which indicates that the fertilization holes are not closed all the time, and the fertilization holes are closed once the clear water activates the sperms to complete fertilization.

The technical solutions provided by the present application are described in detail below with reference to examples, but they should not be understood as limiting the scope of the present application.

### EXAMPLE 1

Tilapia was taken as an experimental subject, and the introduced gene, which was a artificially designed polylysine gene, was set forth in SEQ ID No. 1. The specific sequence was as follows:

ATGAAGAAGAAGAAGAAGAAGAAGAAGAAGAAGAAGAAGAAGAAGAAGAA
GAAGAAGAAGAAGAAGAAGAAGAAGAAGAAGAAGAAGAAGcaccaccacc
accaccacTGATGATAAGAGTGA.

After the sequence was artificially synthesized, it was cloned into pcDNA3.1 expression vector.

Two pairs of primers were designed based on the sequence of pcDNA3.1 expression vector. In the first step of the reaction, primers (SEQ ID No. 2: gcttagggttaggcgttttgcgc; SEQ ID No. 3: gcgcaaaacgcctaaccctaagc) were used for amplification (the amplification product contained the sequence from CMV promoter to the downstream of the tail). The reaction system was 50 μl, which contained 25 μl of 2× Mastermix, 5 μl of primers, 18 μl of ultrapure water, and 2 μl of mutant genomic DNA. The amplification procedure was as follow: pre-denaturing at 95° C. for 2 min, 33 cycles (denaturing at 95° C. for 30 s, annealing at 50° C. for 45 s, extending at 72° C. for 2 min); extending at 72° C. for 5 min.

Four mature male and female tilapia fish were chosen and cultured in a water tank at 29° C. The female fish in estrus was identified through observation, and mature fish eggs were manually squeezed out, and the fish eggs could not touch the water. 4 ml of soaking solution (in the solution, water was taken as a solvent, and each liter contained 6 g of sodium chloride, 0.15 g of potassium chloride, 0.1 g of calcium chloride, 0.1 g of sodium bicarbonate, 0.1 g of sodium dihydrogen phosphate and 1 g of glucose) was taken, 400 μl of cationic liposome Lipofecter was added, then 80 μl of the above PCR product was added, and the above substances were mixed evenly. Then, the mixture was mixed with 1000 fish eggs, and slowly oscillated at 60 rpm for 20 min. And then the sperm was added and mixed, and clear water was added to activate the sperm, thereby completing the fertilization process.

The fertilized eggs were hatched in a natural state.

5 newly hatched small fish were taken, and incubated with 500 μl of STE and 5 μl of proteinase K at 55° C. for 4 hours. After cracking completely, 500 μl solution of phenol:chloroform (1:1) was added, the lysate was vortex oscillated at 12000 g, and centrifuged for 10 min, and the supernatant was taken; 2.5 folds of anhydrous ethanol was added, and centrifuged at 12000 g for 10 min after being mixed with the supernatant evenly; the supernatant was discarded, and 1 ml of 75% ethanol was added into the remaining precipitates, and the precipitates were mixed and centrifuged at 12,000 g for 10 min; the supernatant was discarded and the precipitates were dried in a fume hood. The dried precipitates were added with 50 μl of ultrapure water and dissolved at 37° C.

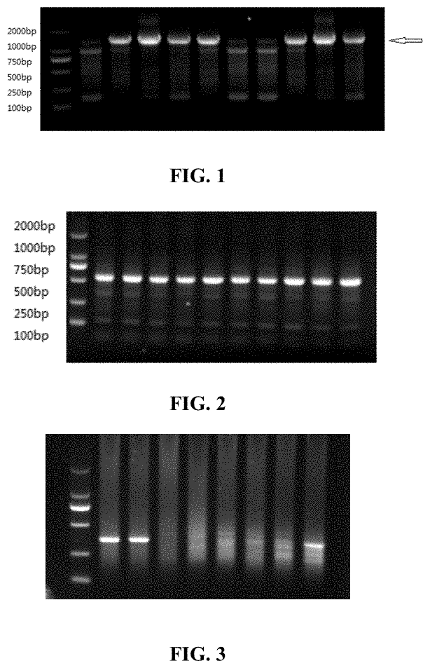

In the first step, primers (SEQ ID NO.4: gcttagggttaggcgttttgcgc; SEQ ID No: 5: agcatgcctgctattgtctt) were used for amplification. The reaction system was 50 μl, which contained 25 μl of 2× mastermix, 5 μl of primer, 18 μl of ultrapure water and 2 μl of mutant genomic DNA. The amplification procedure was: pre-denaturing at 95° C. for 2 min, 33 cycles (denaturing at 95° C. for 30 s, annealing at 50° C. for 45 s, extending at 72° C. for 2 min); extending at 72° C. for 5 min. In the second step, primers (SEQ ID No: 6: ttttggcagtacatcaatgg; SEQ ID No: 7: cagacaatgcgatgcaatttc) were used for amplification (the amplification product contained the target gene fragment) (5 μM). The reaction system was 50 μl, which contained 25 μl of 2× Mastermix (Novomyx), 5 μl of primer, 18 μl of ultrapure water, and 2 μl of PCR product of the first step. The amplification procedure was: pre-denaturing at 95° C. for 2 min, 33 cycles (denaturing at 95° C. for 30 s, annealing at 50° C. for 45 s, extending at 72° C. for 2 min); extending at 72° C. for 5 min. The results were shown in FIG. 1 and FIG. 2.

The band indicated by the arrow in FIG. 1 is the target band, since the number of the introduced sequence entering the fish eggs varies among individuals, and the insertion site are also different, the amplification efficiency of different samples is not completely the same. FIG. 2 is an amplification pattern using the first amplified product as a template, in which the brightest band is the amplified product containing the target fragment, and it can be seen that almost all samples contain the target bands.

### EXAMPLE 2

Carp was taken as an experimental subject, and the introduced gene, which was a small DANA retroposon of zebrafish, was set forth in SEQ ID No.8. The specific nucleotide sequence was as follows:

ggcgacacagtggcgcagtaggtagcacgattgcctcacagcaagaagat
cgctggttcgagtctcggctgggtcagttggcatttctgtgtggagtttg
catgttctcgccgtgttcgcatgggtttcctccgggtgctctggtttccc
ccacagtccaaagacatgcggtacaagtgaattgggtaggctaaattgtt
cgtagtgtatgtgtgtgaatgggagtgtattggcatttcccattgatggg
ttgcagctggaagggcatccgctgcgtaaaagatatgctggaaaagttgg
tggttcattgggctgtggcgaccccagaataataaagggactaagccaaa
aagaaaaaa.

The target gene was constructed into a pEB-GFP(T₂A)PURO lentiviral expression vector. The E. coli XL1-BLUE MRF′ competent cells were used for transformation, the positive clones were selected, and followed by bacterial culture, plasmid extraction and linearization by restriction enzyme SphI.

One mature male and one mature female carp were chosen, injected with a dosage of 40 mg/kg chorionic gonadotropin for fish and cultured in a water tank at room temperature. The mature fish eggs were manually squeezed out next morning. 4 mL of the soaking solution (in the soaking solution, water was taken as a solvent, and each liter contained 6 g of sodium chloride, 0.15 g of potassium chloride, 0.1 g of calcium chloride, 0.1 g of sodium bicarbonate, 0.1 g of sodium dihydrogen phosphate and 1 g of glucose) was taken, 400 μl of the cationic liposome was added, and then 4 μg of the above linearized plasmid was added, and the mixture was mixed evenly. Then, the mixture was mixed with 1000 fish eggs, slowly oscillated at 60 rpm for 20 min. And then the sperm was added, mixed evenly and oscillated gently for several minutes, and the clear water was added to activate the sperm, thereby completing the fertilization process. The fertilized eggs were hatched in a natural state.

8 of the newly hatched small fish were taken and incubated with 500 μl STE and 5 μl Protease K at 55° C. for 4 hours. After cracking completely, 500 μl solution of phenol: chloroform (1:1) was added, the lysate was vortex oscillated at 12,000 g, centrifuged for 10 min, the supernatant was taken; 2.5 folds of anhydrous ethanol was added, and centrifuged at 12,000 g for 10 min after being mixed with the supernatant evenly; The supernatant was discarded, and 1 ml of 75% ethanol was added into the remaining precipitates, and the precipitates were mixed and centrifuged at 12,000 g for 10 min; The supernatant was discarded and the precipitates were dried in a fume hood. The dried precipitates were added with 50 μl of ultrapure water and dissolved at 37° C.

Primers (SEQ ID NO: 9: ttttttctttttggcttagtc; SEQ ID NO: 10: ggcgacacagtggcgcagta) were used for amplification. The reaction system was 50 μl, which contained 25 μl of 2× Mastermix, 5 μl of primer, 18 μl of ultrapure water, and 2 μl of mutant genomic DNA. The amplification procedure was: pre-denaturing at 95° C. for 2 min, 33 cycles (denaturing at 95° C. for 30 s, annealing at 50° C. for 45 s, extending at 72° C. for 2 min); extending at 72° C. for 5 min. The results were shown in FIG. 3.

The amplified pattern of the DANA retroposon can be seen from FIG. 3, the length of DANA retroposon is 359 bp, and three of the eight samples have obvious amplified bands, three have unobvious amplified bands, and two have no amplified bands, indicating that three of the eight samples have confirmed target bands, that is, the target sequences have entered the sample genome; two are negative, indicating that the sample has no target sequence; three have insignificant amplified bands, indicating that the target sequences are degraded to some degrees after entering the samples.

The above described are only preferred embodiments of the present application, it should be understood by those skilled in the art that, without departing from the principle of the present application, several improvements and modifications can be made, and these improvements and modifications also should be regarded as the protection scope of the present application fall into the scope of the present application.

What is claimed is:

1. A solution for prolonging open time of fertilization hole of fish eggs, wherein the solution takes water as a solvent, and comprises 6 g of sodium chloride, 0.15 g of potassium chloride, 0.1 g of calcium chloride, 0.1 g of sodium bicarbonate, 0.1 g of sodium dihydrogen phosphate and 1 g of glucose per liter.

2. The solution according to claim 1, wherein the water is distilled water.

3. A method for introducing exogenous gene through fertilization hole, comprising the steps of:
1) mixing the solution in claim 1 with the cationic liposome and exogenous genes to obtain a mixed solution;
2) mixing the mixed solution obtained in the step 1) with fish eggs, and oscillating for 10˜30 min.

4. The method according to claim 3, wherein the volume ratio of the solution to the cationic liposome in step 1) is 1:0.1.

5. The method according to claim 3, wherein the ratio of the volume of the mixed solution to the number of the fish eggs in step 2) is 4 ml:1000.

6. The method according to claim 3, wherein the oscillating time in step 2) is 20 min.

7. The method according to claim 3, wherein the oscillating speed in step 2) is 60 rpm.

8. The method according to claims 3, wherein the fish eggs are selected from eggs of tilapia and carp.

9. The method according to claim 5, wherein the fish eggs are selected from eggs of tilapia and carp.

10. The solution according to claim 1, wherein the fish eggs are selected from eggs of tilapia and carp.

11. The method according to claim 4, wherein the cationic liposome is Lipofecter.


SEQUENCE LISTING (10 sequences)

SEQ ID NO: 1 - DNA, 123 bp, artificial sequence
  artificially designed polylysine gene
atgaagaagaagaagaagaagaagaagaagaagaagaagaagaagaagaagaagaagaagaagaagaagaagaagaagaagaagaagaagcaccaccaccaccaccactgatgataagagtga

SEQ ID NO: 2 - DNA, 23 bp, artificial sequence
  primer in the first step of the reaction
gcttagggttaggcgttttgcgc

SEQ ID NO: 3 - DNA, 23 bp, artificial sequence
  primer in the first step of the reaction
gcgcaaaacgcctaaccctaagc

SEQ ID NO: 4 - DNA, 23 bp, artificial sequence
  primer in the first step
gcttagggttaggcgttttgcgc

SEQ ID NO: 5 - DNA, 20 bp, artificial sequence
  primer in the first step
agcatgcctgctattgtctt

SEQ ID NO: 6 - DNA, 20 bp, artificial sequence
  primer in the second step
ttttggcagtacatcaatgg

SEQ ID NO: 7 - DNA, 21 bp, artificial sequence
  primer in the second step
cagacaatgcgatgcaatttc

SEQ ID NO: 8 - DNA, 359 bp, artificial sequence
  small DANA retroposon of zebrafish
ggcgacacagtggcgcagtaggtagcacgattgcctcacagcaagaagatcgctggttcgagtctcggctgggtcagttggcatttctgtgtggagtttgcatgttctcgccgtgttcgcatgggtttcctccgggtgctctggtttcccccacagtccaaagacatgcggtacaagtgaattgggtaggctaaattgttcgtagtgtatgtgtgtgaatgggagtgtattggcatttcccattgatgggttgcagctggaagggcatccgctgcgtaaaagatatgctggaaaagttggtggttcattgggctgtggcgaccccagaataataaagggactaagccaaaaagaaaaaa

SEQ ID NO: 9 - DNA, 21 bp, artificial sequence
  Primer
ttttttctttttggcttagtc

SEQ ID NO: 10 - DNA, 20 bp, artificial sequence
  Primer
ggcgacacagtggcgcagta